(19) United States
(12) Patent Application Publication
(10) Pub. No.: US 2026/0224650 A1
(43) Pub. Date: Aug. 6, 2026

(54) GENE THERAPY FOR TREATING PHENYLKETONURIA

(71) Applicant: The Trustees of the University of Pennsylvania, Philadelphia, PA (US)

(72) Inventors: James M. Wilson, Philadelphia, PA (US); Jenny Agnes Sidrane, Phoenixville, PA (US); Scott Ashley, Philadelphia, PA (US)

(21) Appl. No.: 19/535,513

(22) Filed: Feb. 10, 2026

Related U.S. Application Data
(63) Continuation of application No. 17/832,961, filed on Jun. 6, 2022, now Pat. No. 12,569,525.
Application No. 17/832,961 is a continuation of application No. 16/474,960, filed on Dec. 29, 2017, now Pat. No. 11,382,941.
(60) Provisional application No. 62/440,651, filed on Dec. 30, 2016.
(60) Provisional application No. 62/469,898, filed on Mar. 10, 2017.
(60) Provisional application No. 62/505,373, filed on May 12, 2017.

(51) Int. Cl. A61K 35/761; A61K 48/00; A61P 3/00; C12N 15/52; C12N 15/86

(52) U.S. Cl. CPC A61K 35/761; A61K 48/0075; A61K 48/0083; A61P 3/00; C12N 15/52; C12N 15/86; C12Y 114/16001

(57) ABSTRACT

Compositions and regimens useful in treating phenylketonuria are provided. The compositions include recombinant adeno-associated virus (rAAV) with a transthyretin enhancer and promoter driving expression of a human phenylalanine hydroxylase.

### CROSS-REFERENCE TO RELATED APPLICATIONS

This application is a continuation of U.S. patent application Ser. No. 17/832,961, filed Jun. 6, 2022, which is a continuation of U.S. patent application Ser. No. 16/474,960, filed Jun. 28, 2019, now U.S. Pat. No. 11,382,941, issued Jul. 12, 2022, which is a national stage application under 35 U.S.C. § 371 of International Patent Application No. PCT/US2017/068897, filed Dec. 29, 2017, which claims the benefit under 35 U.S.C. § 119 (e) of U.S. Provisional Patent Application No. 62/440,651, filed Dec. 30, 2016; U.S. Provisional Patent Application No. 62/469,898, filed Mar. 10, 2017; and U.S. Provisional Patent Application No. 62/505,373, filed May 12, 2017. The foregoing applications are incorporated by reference herein in their entireties.

### INCORPORATION-BY-REFERENCE OF MATERIAL SUBMITTED IN ELECTRONIC FORM

The contents of the electronic sequence listing (UPN-16-7939USC2_20260209_SeqListing.xml; Size: 64,100 bytes; Date of Creation: Feb. 9, 2026) are incorporated by reference herein in its entirety.

### 1. INTRODUCTION

The application relates to embodiments useful for a gene therapy for treating phenylketonuria.

### 2. BACKGROUND

As one of the most common inborn errors of metabolism, Phenylketonuria (PKU) occurs in 1 in 10,000 to 15,000 newborns in the United States. The current treatment approaches require the affected individual to adhere consistently to an unpalatable and expensive dietary restriction and/or take enzyme substitution with phenylalanine ammonia lyase from birth for their whole life.

The most common cause of PKU is deficiency of phenylalanine hydroxylase (PAH) due to a recessively inherited mutation in the PAH gene. PAH is expressed primarily in the liver that catalyzes the irreversible hydroxylation of phenylalanine to tyrosine. Thus, deficiency in PAH affects the catabolic pathway of phenylalanine, resulting in accumulation of phenylalanine. High plasma phenylalanine levels results in build-up of phenylalanine in the brain and can affect brain development and function, resulting in intellectual disability and seizures. Furthermore, reduction of plasma phenylalanine via dietary restriction and enzyme substitution is expensive, inconvenient and has been linked with various adverse complications, such as persistent mild cognitive deficits.

An alternative approach to achieve sustained therapeutic levels of PAH is through continuous in vivo production of the native enzyme in the hepatocytes using gene transfer mediated by a cell-directed adeno-associated virus (AAV) or other viral or non-viral vector. Several attempts of vector-mediated PAH expression have been tested preliminary on mouse studies. See, e.g., Harding et al, Complete correction of hyperphenylalaninemia following liver-directed, recombinant AAV2/8 vector mediated gene therapy in murine phenylketonuria Gene Ther. 2006 March; 13(5):457-6 and Viecelli et al, Treatment of Phenylketonuria Using Minicircle-Based Naked-DNA Gene Transfer to Murine Liver Hepatology. 2014 September; 60(3): 1035-1043, which are incorporated herein by reference. However, the evaluations of delivery efficiency, immune stimulation, long-term expression stability and safety are either lacking or not optimal. Thus, more efficient AAV.hPAH vectors are needed for PKU treatment.

### SUMMARY

The embodiments described herein relate to an AAV gene therapy vector for delivering normal human phenylalanine hydroxylase (PAH) to a subject in need thereof, following intravenous administration of the vector resulting in long-term, perhaps 10 years or more, of clinically meaningful correction of hyperphenylalaninemia. The subject patient population is patients with moderate to severe hyperphenylalaninemia, including those with PKU, variant PKU or non-PKU hyperphenylalaninemia. The intended vector dose is intended to deliver PAH blood levels of approximately 15% or greater as compared to wild type, which is the level which has been reported for “moderate” PKU patients. See, Kaufman, S., PNAS, 96:3160-4 (1999), which is incorporated herein by reference. In another embodiment, the intended vector dose is intended to deliver PAH to result in a reduction of plasma phenylalanine levels by 25% or greater. In one embodiment, the goal for the AAV vector treatment is conversion of severe PKU patients to either moderate or mild PKU thus lessening the burden associated with a severely limited phenylalanine diet.

In one aspect, this application provides the use of a replication deficient adeno-associated virus (AAV) to deliver a human phenylalanine hydroxylase (PAH) gene to liver cells of patients (human subjects) diagnosed with PKU. The recombinant AAV vector (rAAV) used for delivering the hPAH gene (“rAAV.hPAH”) should have a tropism for the liver (e.g., a rAAV bearing an AAV8 capsid), and the hPAH transgene should be controlled by liver-specific expression control elements. In one embodiment, the expression control elements include one or more of the following: an enhancer; a promoter; an intron; a WPRE; and a polyA signal. Such elements are further described herein.

In one embodiment, the hPAH coding sequence is shown in SEQ ID NO: 1. In one embodiment, the PAH protein sequence is shown in SEQ ID NO: 2. The coding sequence for hPAH is, in one embodiment, codon optimized for expression in humans. Such sequence may share less than 80% identity to the native hPAH coding sequence (SEQ ID NO: 3). In one embodiment, the hPAH coding sequence is that shown in SEQ ID NO: 1.

In another aspect, provided herein is an aqueous suspension suitable for administration to a PKU patient which includes the rAAV described herein. In some embodiments, the suspension includes an aqueous suspending liquid and about 1×10¹² to about 1×10¹⁴ genome copies (GC) of the rAAV/mL. The suspension is, in one embodiment, suitable for intravenous injection. In other embodiment, the suspension further includes a surfactant, preservative, and/or buffer dissolved in the aqueous suspending liquid.

In another embodiment, provided herein is a method of treating a patient having PKU with an rAAV as described herein. In one embodiment, about 1×10¹¹ to about 3×10¹³ genome copies (GC) of the rAAV/kg patient body weight are delivered the patient in an aqueous suspension.

### 3. BRIEF DESCRIPTION OF THE DRAWINGS

FIG. 1 is a schematic representation of pAAV.TBG.PI.hPAHco.WPRE.bGH cis plasmid.

FIG. 2A is a bar graph of plasma phenylalanine (Phe) levels in PAH_KO_A mouse model (shown in white) and wild-type (shown in black) or heterozygous (shown in grey) littermates, as described in Example 1. These results are summarized in FIG. 2D.

FIG. 2B is a line graph of plasma phenylalanine (Phe) levels in PAH_KO_A mice (shown in white) with heterozygous (shown in grey) and wild-type (shown in black) littermates provided as controls. Mice were injected with 1×10¹³ GC/kg or 1×10¹² GC/kg of AAV8.TBG.PI.hPAHco.WPRE.bGH on day 56 of natural history study as described in Example 3. Experiment was performed on 7 male and 3 female PAH_KO_A mice from the natural history study.

FIG. 2C is a line graph of plasma phenylalanine (Phe) levels in PAH_KO_A mouse model injected with 1×10¹³ GC/kg or 1×10¹² GC/kg of AAV8.TBG.PI.hPAHco. WPRE.bGH as described in Example 3. The day of injection was Day 0.

FIG. 2D is a line graph showing the mean Phe levels for the mice studied in FIG. 2A. Values expressed as mean±/−SEM.

FIG. 3A is a bar graph of plasma phenylalanine (Phe) levels in PAH_KO_B mouse model (shown in white) and wild-type (shown in black) or heterozygous (shown in grey) littermates, as described in Example 1. These results are summarized in FIG. 3D.

FIG. 3B is a line graph of plasma phenylalanine (Phe) levels in PAH_KO_B mice (shown in white) with heterozygous (shown in grey) and wild-type (shown in black) littermates provided as controls. Experiment was performed on 3 female PAH_KO_B mice from the natural history study.

FIG. 3C is a line graph of plasma phenylalanine (Phe) levels in PAH_KO_B mouse model injected with 1×10¹² GC/kg of AAV8.TBG.PI.hPAHco. WPRE.bGH as described in Example 3. Mice Identification Numbers 1691, 1695 and 1696 were females injected with 1×10¹² GC/kg on Day 0.

FIG. 3D is a line graph showing the mean Phe levels for the mice studied in FIG. 3A. Values expressed as mean±/−SEM.

FIG. 4A is a line graph of plasma phenylalanine (Phe) levels in PAH_KO_C mouse model (shown in white) and wild-type (shown in black) or heterozygous (shown in grey) littermates, as described in Example 1. These results are summarized in FIG. 4C.

FIG. 4B is a line graph of plasma phenylalanine (Phe) levels in PAH_KO_C mice (shown in white) with heterozygous (shown in grey) and wild-type (shown in black) littermates provided as controls. Experiment was performed on 2 male PAH_KO_C mice from the natural history study.

FIG. 4C is a line graph showing the mean Phe levels for the mice studied in FIG. 4A. Values expressed as mean±/−SEM.

FIG. 5 is a bar graph summarizing the results of FIGS. 2A, 3A and 4A. Plasma phenylalanine (Phe) levels were detected via LC/MS/MS and the data from PAH_KO_A, PAH_KO_B and PAH_KO_C mice bled from 6-8 weeks of age. Plasma was isolated and analyzed for Phe concentration. Wild-type littermates were provided as negative controls.

FIG. 6A-6C demonstrate that AAV8.TBG.hPAHco rescues phenylalanine levels in PKU_KO_B mice. PKU B mice ages 17-22 weeks were given either 10¹² GC/kg of either AAV8.TBG.hPAHco.bGH (circles) or AAV8.TBG.hPAHco. WPRE.bGH (squares) after pretreatment phenylalanine levels were established. PBS treatment shown with triangles. Mice were then bled weekly, and plasma was isolated and phenylalanine concentration was determined (FIG. 6A). At the termination of the study, liver was collected and genome copy analysis (FIG. 6B) and immunohistochemistry (FIG. 6C) was performed. Values expressed as mean±SEM.

FIG. 7 is a line graph demonstrating that AAV gene therapy lowers plasma Phe concentration in PKU_KO_A mice. WT (triangle), heterozygous (circle) and PKU_KO_A (KO) mice were injected intravenously with 10¹¹ GC/kg of AAV8.TBG.hPAHco after baseline phenylalanine levels were established. Mice were then bled weekly, and plasma was isolated and analyzed for Phe concentration. Values expressed as mean±/−SEM. Phe levels in plasma decreased by 71% following intravenous administration of AAV8.TBG.hPAHco.

FIGS. 8A-8C demonstrate that high does AAV9.TBG.hPAHco rescues phenylalanine levels in PKU_KO_B mice. PKU_KO_B mice were given either 10¹² GC/kg, 3×10¹¹ GC/kg, or 10¹¹ GC/kg, of AAV8.TBG.hPAHco after baseline phenylalanine levels were established. Mice were then bled weekly, and plasma was isolated and analyzed for Phe concentration (FIG. 8A). Values expressed as mean±/−SEM. At termination of the study, liver was collected and genome copy analysis (FIG. 8B) and immunohistochemistry (FIG. 8C) was performed. Protein expression and reduction in Phe levels seen at a dose of 10¹² GC/kg.

FIG. 9 shows an alignment of a portion of the PAH sequence for wild type (WT), PAH_KO_A (Strain A), PAH_KO_B (Strain B), PAH_KO_C (Strain C), PAH_KO_D (Strain D) and consensus.

### 4. DETAILED DESCRIPTION

The embodiments described in the application relate to the use of a replication deficient adeno-associated virus (AAV) to deliver a human phenylalanine hydroxylase (PAH) gene to liver cells of patients (human subjects) diagnosed with phenylketonuria (PKU). The recombinant AAV vector (rAAV) used for delivering the hPAH gene (“rAAV.hPAH”) should have a tropism for the liver (e.g., a rAAV bearing an AAV8 capsid), and the hPAH transgene should be controlled by liver-specific expression control elements. In one embodiment, the expression control elements include one or more of the following: an enhancer; a promoter; an intron; a WPRE; and a polyA signal. Such elements are further described herein.

As used herein, “AAV8 capsid” refers to the AAV8 capsid having the amino acid sequence of GenBank, accession: YP_077180.1, SEQ ID NO: 19, which is incorporated by reference herein. Some variation from this encoded sequence is permitted, which may include sequences having about 99% identity to the referenced amino acid sequence in YP_077180.1 and WO 2003/052051 (which is incorporated herein by reference) (i.e., less than about 1% variation from the referenced sequence). Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2015/0315612.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 32 amino acids, about 330 amino acids, or a peptide fragment thereof or the corresponding nucleic acid sequence coding sequencers. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, the term “operably linked” refers to both expression control sequences that are contiguous with the gene of interest and expression control sequences that act in trans or at a distance to control the gene of interest.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### 5.1 Gene Therapy Vectors

In one aspect, a recombinant adeno-associated virus (rAAV) vector carrying the human PAH gene is provided for use in gene therapy. The rAAV.hPAH vector should have a tropism for the liver (e.g., a rAAV bearing an AAV8 capsid) and the hPAH transgene should be controlled by liver-specific expression control elements. The vector is formulated in a buffer/carrier suitable for infusion in human subjects. The buffer/carrier should include a component that prevents the rAAV from sticking to the infusion tubing but does not interfere with the rAAV binding activity in vivo.

5.1.1. The rAAV.hPAH Vector
5.1.1.1. The hPAH Sequence

Phenylketonuria is an inherited error of metabolism caused predominantly by mutations in the phenylalanine hydroxylase (PAH) gene. Mutations in the PAH gene result in decreased catalytic activity affecting the catabolic pathway of phenylalanine (Phe). PAH is a hepatic enzyme that requires the cofactor tetrahydrobiopterin (BH4) to convert Phe to tyrosine (Tyr). A deficiency in PAH or its cofactor BH4 results in the accumulation of excess phenylalanine, whose toxic effects can cause severe and irreversible intellectual disability and other disorders, if untreated. See, Havid and Cristodoulou, Transl Pediatr, 2015 Oct. 4(4):304-17, which is incorporated herein by reference.

Over 550 mutations of the PAH gene have been described, the majority of which result in deficient enzyme activity. See, Phenylalanine Hydroxylase Locus Knowledgebase, accessed online at pahdb.mcgill.ca/, which is incorporated herein by reference. Due to the large number of known PKU mutations, and the autosomal recessive nature of the disease, a wide range of disease severity is seen. The severity of the disease is generally classified by blood phenylalanine levels, which are sometimes classified as classic PKU, moderate or variant PKU, mild PKU, or hyperphenylalaninemia. Based on blood Phe levels at diagnosis, there are 4 levels of PKU severity.
Hyperphenylalaninemia, with Phe levels that are slightly above normal range: 120-600 μmol/L (2-10 mg/dL)
Mild, with the lowest blood Phe levels: 600-900 μmol/L (10-15 mg/dL)
Moderate or variant, with blood Phe levels somewhere in the middle: 900-1200 μmol/L (15-20 mg/dL)
Severe or “classic” PKU, with extremely high blood Phe levels: >1200 μmol/L (20 mg/dL)

The goal of therapies described herein would provide functional PAH enzyme resulting in Phe levels in the 120-600 μmol/L range, e.g., a 25% or greater reduction in plasma Phe levels. In another embodiment, the vector dose is intended to deliver PAH to result in a reduction of plasma phenylalanine levels by 25% or greater. In another embodiment, the vector dose is intended to deliver PAH to result in a reduction of plasma phenylalanine levels by 30% or greater. In another embodiment, the vector dose is intended to deliver PAH to result in a reduction of plasma phenylalanine levels by 35% or greater. In another embodiment, the vector dose is intended to deliver PAH to result in a reduction of plasma phenylalanine levels by 40% or greater. In another embodiment, the vector dose is intended to deliver PAH to result in a reduction of plasma phenylalanine levels by 45% or greater. In another embodiment, the vector dose is intended to deliver PAH to result in a reduction of plasma phenylalanine levels by 50% or greater. In another embodiment, the vector dose is intended to deliver PAH to result in a reduction of plasma phenylalanine levels by 60% or greater. In another embodiment, the vector dose is intended to deliver PAH to result in a reduction of plasma phenylalanine levels by 70% or greater. In another embodiment, the vector dose is intended to deliver PAH to result in a reduction of plasma phenylalanine levels by 75% or greater.

In one embodiment, the “subject” or “patient” is a mammalian subject having PKU as described above. It is intended that a patient having PKU of any severity is the intended subject.

In one embodiment, the hPAH gene encodes the hPAH protein shown in SEQ ID NO: 2. Thus, in one embodiment, the hPAH transgene can include, but is not limited to, the sequence provided by SEQ ID NO: 1 or SEQ ID NO: 3 which are provided in the attached Sequence Listing, which is incorporated by reference herein. SEQ ID NO: 3 provides the cDNA for native human PAH. SEQ ID NO: 1 provides an engineered cDNA for human PAH, which has been codon optimized for expression in humans (sometimes referred to herein as hPAHco). It is to be understood that reference to hPAH herein may, in some embodiments, refer to the hPAH native or codon optimized sequence. Alternatively or additionally, web-based or commercially available computer programs, as well as service based companies may be used to back translate the amino acid sequences to nucleic acid coding sequences, including both RNA and/or cDNA. See, e.g., backtranseq by EMBOSS, http://www.ebi.ac.uk/Tools/st/; Gene Infinity (http://www.geneinfinity.org/sms-/sms_backtranslation.html); ExPasy (http://www.expasy.org/tools/). It is intended that all nucleic acids encoding the described hPAH polypeptide sequences are encompassed, including nucleic acid sequences which have been optimized for expression in the desired target subject (e.g., by codon optimization).

In one embodiment, the nucleic acid sequence encoding hPAH shares at least 95% identity with the native hPAH coding sequence of SEQ ID NO: 3. In another embodiment, the nucleic acid sequence encoding hPAH shares at least 90, 85, 80, 75, 70, or 65% identity with the native hPAH coding sequence of SEQ ID NO: 3. In one embodiment, the nucleic acid sequence encoding hPAH shares about 78% identity with the native hPAH coding sequence of SEQ ID NO: 3. In one embodiment, the nucleic acid sequence encoding hPAH is SEQ ID NO: 1.

In one embodiment, the PAH coding sequence is optimized for expression in the target subject. Codon-optimized coding regions can be designed by various different methods. This optimization may be performed using methods which are available on-line (e.g., GeneArt), published methods, or a company which provides codon optimizing services, e.g., as DNA2.0 (Menlo Park, CA). One codon optimizing approach is described, e.g., in International Patent Publication No. WO 2015/012924, which is incorporated by reference herein. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Thermo Fisher Scientific Inc. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

5.1.1.2. The rAAV Vector

Because PAH is natively expressed in the liver, it is desirable to use an AAV which shows tropism for liver. In one embodiment, the AAV supplying the capsid is AAV8. In another embodiment, the AAV supplying the capsid is AAVrh. 10. In yet another embodiment, the AAV supplying the capsid is a Clade E AAV. Such AAV include rh.2; rh. 10; rh. 25; bb.1, bb.2, pi.1, pi.2, pi.3, rh.38, rh.40, rh.43, rh.49, rh.50, rh.51, rh.52, rh.53, rh.57, rh.58, rh.61, rh.64, hu.6, hu. 17, hu.37, hu.39, hu.40, hu.41, hu.42, hu.66, and hu.67. This clade further includes modified rh. 2; modified rh. 58; and modified rh.64. See, WO 2005/033321, which is incorporated herein by reference. However, any of a number of rAAV vectors with liver tropism can be used.

In a specific embodiment described in the Examples, infra, the gene therapy vector is an AAV8 vector expressing an hPAH transgene under control of a thyroxine binding globulin (TBG) promoter referred to as AAV8.TBG.PI.hPAHco. WPRE.bGH. The vector genome for such a vector is shown in SEQ ID NO: 20. In another embodiment, the WPRE is omitted, i.e., AAV8.TBG.PI.hPAHco.bGH. The vector genome for such a vector is shown in SEQ ID NO: 21. The external AAV vector component is a serotype 8, T=1 icosahedral capsid consisting of 60 copies of three AAV viral proteins, VP1, VP2, and VP3, at a ratio of 1:1:10. The capsid contains a single-stranded DNA rAAV vector genome.

In one embodiment, the rAAV.hPAH genome contains an hPAH transgene flanked by two AAV inverted terminal repeats (ITRs). In one embodiment, the hPAH transgene includes one or more of an enhancer, promoter, an intron, a Woodchuck Hepatitis Virus (WHP) Posttranscriptional Regulatory Element (WPRE) (e.g., SEQ ID NO: 15), an hPAH coding sequence, and polyadenylation (polyA) signal. In another embodiment, the hPAH transgene includes one or more of an enhancer, promoter, an intron, an hPAH coding sequence, and polyadenylation (polyA) signal. These control sequences are “operably linked” to the hPAH gene sequences. The expression cassette containing these sequences may be engineered onto a plasmid which is used for production of a viral vector.

The ITRs are the genetic elements responsible for the replication and packaging of the genome during vector production and are the only viral cis elements required to generate rAAV. The minimal sequences required to package the expression cassette into an AAV viral particle are the AAV 5′ and 3′ ITRs, which may be of the same AAV origin as the capsid, or which of a different AAV origin (to produce an AAV pseudotype). In one embodiment, the ITR sequences from AAV2, or the deleted version thereof (ΔITR), are used. However, ITRs from other AAV sources may be selected. Where the source of the ITRs is from AAV2 and the AAV capsid is from another AAV source, the resulting vector may be termed pseudotyped. Typically, an expression cassette for an AAV vector comprises an AAV 5′ ITR, the hPAH coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used. In one embodiment, the 5′ ITR is that shown in SEQ ID NO: 16. In one embodiment, the 3′ ITR is that shown in SEQ ID NO: 17.

In one embodiment, the expression control sequences include one or more enhancer. In one embodiment, the En34 enhancer is included (34 bp core enhancer from the human apolipoprotein hepatic control region), which is shown in SEQ ID NO: 4. In another embodiment, the EnTTR (100 bp enhancer sequence from transthyretin) is included. Such sequence is shown in SEQ ID NO: 5. See, Wu et al, Molecular Therapy, 16(2): 280-289, February 2008, which is incorporated herein by reference. In yet another embodiment, the α1-microglogulin/bikunin precursor enhancer is included. In yet another embodiment, the ABPS (shortened version of the 100 bp distal enhancer from the al-microglogulin/bikunin precursor [ABP] to 42 bp) enhancer is included. Such sequence is shown in SEQ ID NO: 6. In yet another embodiment, the ApoE enhancer is included. Such sequence is shown in SEQ ID NO: 7. In another embodiment, more than one enhancer is present. Such combination may include more than one copy of any of the enhancers described herein, and/or more than one type of enhancer.

Expression of the hPAH coding sequence is driven from a liver-specific promoter. An illustrative plasmid and vector described herein uses the thyroxine binding globulin (TBG) promoter (SEQ ID NO: 9), or a modified version thereof. One modified version of the TBG promoter is a shortened version, termed TBG-S1. A modified thyroxine binding globulin (TBG-S1) promoter sequence is shown in SEQ ID NO: 8. Alternatively, other liver-specific promoters may be used such as the transthyretin promoter. Another suitable promoter is the alpha 1 anti-trypsin (A1AT) promoter, or a modified version thereof (which sequence is shown in SEQ ID NO: 10). Another suitable promoter is the TTR promoter (SEQ ID NO: 11). Other suitable promoters include human albumin (Miyatake et al., J. Virol., 71:5124 32 (1997)), humAlb; the Liver Specific promoter (LSP), and hepatitis B virus core promoter, (Sandig et al., Gene Ther., 3:1002 9 (1996). See, e.g., The Liver Specific Gene Promoter Database, Cold Spring Harbor, available online at rulai.schl.edu/LSPD, which is incorporated by reference. Although less desired, other promoters, such as viral promoters, constitutive promoters, regulatable promoters [see, e.g., WO 2011/126808 and WO 2013/04943], or a promoter responsive to physiologic cues may be used may be utilized in the vectors described herein.

In addition to a promoter, an expression cassette and/or a vector may contain other appropriate transcription initiation, termination, enhancer sequences, and efficient RNA processing signals. Such sequences include splicing and polyadenylation (polyA) signals; regulatory elements that enhance expression (i.e., WPRE (SEQ ID NO: 15)); sequences that stabilize cytoplasmic mRNA; sequences that enhance translation efficiency (i.e., Kozak consensus sequence); sequences that enhance protein stability; and when desired, sequences that enhance secretion of the encoded product. In one embodiment, a polyadenylation (polyA) signal is included to mediate termination of hPAH mRNA transcripts. Examples of other suitable polyA sequences include, e.g., bovine growth hormone (SEQ ID NO: 12), SV40, rabbit beta globin, and TK polyA, amongst others.

In one embodiment, the regulatory sequences are selected such that the total rAAV vector genome is about 2.0 to about 5.5 kilobases in size. In one embodiment, the regulatory sequences are selected such that the total rAAV vector genome is about 3.4 kb, about 2.9 kb, about 3.3 kb, about 2.2 kb or about 2.5 kb in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al, Mol Ther, January 2010 18(1):80-6, which is incorporated herein by reference.

Thus, in one embodiment, an intron is included in the vector. Suitable introns include the human beta globin IVS2 (SEQ ID NO: 13). See, Kelly et al, Nucleic Acids Research, 43(9):4721-32 (2015), which is incorporated herein by reference. Another suitable promoter includes the Promega chimeric intron (SEQ ID NO: 14), sometimes referred to as “PI”). See, Almond, B. and Schenborn, E. T. A Comparison of pCI-neo Vector and pcDNA4/HisMax Vector. [Internet] 2000, which is incorporated herein by reference. Available from: www.promega.com/resources/pubhub/enotes/a-comparison-of-pcineo-vector-and-pcdna4hismax-vector/). Another suitable intron includes the hFIX intron (SEQ ID NO: 18). Various introns suitable herein are known in the art and include, without limitation, those found at http://bpg.utoledo.edu/~afedorov/lab/eid.html, which is incorporated herein by reference. See also, Shepelev V., Fedorov A. Advances in the Exon-Intron Database. Briefings in Bioinformatics 2006, 7:178-185, which is incorporated herein by reference.

In one embodiment, the rAAV vector genome comprises SEQ ID NO: 20, SEQ ID NO: 21, SEQ ID NO:22, SEQ ID NO: 23, SEQ ID NO: 24 or SEQ ID NO:25.

### 5.1.2. Compositions

In one embodiment, the rAAV.hPAH virus is provided in a pharmaceutical composition which comprises an aqueous carrier, excipient, diluent or buffer. In one embodiment, the buffer is PBS. In a specific embodiment, the rAAV.hPAH formulation is a suspension containing an effective amount of rAAV.hPAH vector suspended in an aqueous solution containing 0.001% Pluronic F-68 in TMN200 (200 mM sodium chloride, 1 mM magnesium chloride, 20 mM Tris, pH 8.0). However, various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

For example, a suspension as provided herein may contain both NaCl and KCl. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension. In another embodiment, the vector is suspended in an aqueous solution containing 180 mM sodium chloride, 10 mM sodium phosphate, 0.001% Poloxamer 188, pH 7.3.

In one embodiment, the formulation is suitable for use in human subjects and is administered intravenously. In one embodiment, the formulation is delivered via a peripheral vein by bolus injection. In one embodiment, the formulation is delivered via a peripheral vein by infusion over about 10 minutes (±5 minutes). In one embodiment, the formulation is delivered via a peripheral vein by infusion over about 20 minutes (±5 minutes). In one embodiment, the formulation is delivered via a peripheral vein by infusion over about 30 minutes (±5 minutes). In one embodiment, the formulation is delivered via a peripheral vein by infusion over about 60 minutes (±5 minutes). In one embodiment, the formulation is delivered via a peripheral vein by infusion over about 90 minutes (±10 minutes). However, this time may be adjusted as needed or desired. Any suitable method or route can be used to administer an AAV-containing composition as described herein, and optionally, to co-administer other active drugs or therapies in conjunction with the AAV-mediated delivery of hPAH described herein. Routes of administration include, for example, systemic, oral, inhalation, intranasal, intratracheal, intraarterial, intraocular, intravenous, intramuscular, subcutaneous, intradermal, and other parental routes of administration.

In one embodiment, the formulation may contain, e.g., about 1.0×10¹¹ genome copies per kilogram of patient body weight (GC/kg) to about 1×10¹⁵ GC/kg, about 5×10¹¹ genome copies per kilogram of patient body weight (GC/kg) to about 3×10¹³ GC/kg, or about 1×10¹² to about 1×10¹⁴ GC/kg, as measured by oqPCR or digital droplet PCR (ddPCR) as described in, e.g., M. Lock et al, Hum Gene Ther Methods. 2014 April; 25(2): 115-25. doi: 10.1089/hgtb.2013.131. Epub 2014 Feb. 14, which is incorporated herein by reference. In one embodiment, the rAAV.hPAH formulation is a suspension containing at least 1×10¹³ genome copies (GC)/mL, or greater, as measured by oqPCR or digital droplet PCR (ddPCR) as described in, e.g., M. Lock et al, supra.

In order to ensure that empty capsids are removed from the dose of AAV.hPAH that is administered to patients, empty capsids are separated from vector particles during the vector purification process, e.g., using the method discussed herein. In one embodiment, the vector particles containing packaged genomes are purified from empty capsids using the process described in U.S. Patent Appln No. 62/322,098, filed on Apr. 13, 2016, and entitled “Scalable Purification Method for AAV8”, which is incorporated by reference herein. Briefly, a two-step purification scheme is described which selectively captures and isolates the genome-containing rAAV vector particles from the clarified, concentrated supernatant of a rAAV production cell culture. The process utilizes an affinity capture method performed at a high salt concentration followed by an anion exchange resin method performed at high pH to provide rAAV vector particles which are substantially free of rAAV intermediates. Similar purification methods can be used for vectors having other capsids.

While any conventional manufacturing process can be utilized, the process described herein (and in U.S. Patent Appln No. 62/322,098) yields vector preparations wherein between 50 and 70% of the particles have a vector genome, i.e., 50 to 70% full particles. Thus for an exemplary dose of 1.6×10¹² GC/kg, and the total particle dose will be between 2.3×10¹² and 3×10¹² particles. In another embodiment, the proposed dose is one half log higher, or 5×10¹² GC/kg, and the total particle dose will be between 7.6×10¹² and 1.1×10¹³ particles. In one embodiment, the formulation is be characterized by an rAAV stock having a ratio of “empty” to “full” of 1 or less, preferably less than 0.75, more preferably, 0.5, preferably less than 0.3.

A stock or preparation of rAAV8 particles (packaged genomes) is “substantially free” of AAV empty capsids (and other intermediates) when the rAAV8 particles in the stock are at least about 75% to about 100%, at least about 80%, at least about 85%, at least about 90%, at least about 95%, or at least 99% of the rAAV8 in the stock and “empty capsids” are less than about 1%, less than about 5%, less than about 10%, less than about 15% of the rAAV8 in the stock or preparation.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacylamide gels (e.g., Novex). Silver staining may be performed using SilverXpress (Invitrogen, CA) according to the manufacturer's instructions. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is provided herein which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

### 5.2 Patient Population

As discussed above, a subject having PKU of any severity is the intended recipient of the compositions and methods described herein.

Subjects may be permitted to continue their standard of care treatment(s) (e.g., diet low in Phe; treatment with sapropterin dihydrochloride) prior to and concurrently with the gene therapy treatment at the discretion of their caring physician. In the alternative, the physician may prefer to stop standard of care therapies prior to administering the gene therapy treatment and, optionally, resume standard of care treatments as a co-therapy after administration of the gene therapy.

Desirable endpoints of the gene therapy regimen are an increase in PAH activity resulting in Phe levels between 120-360 μmol/L. In another embodiment, the vector dose is intended to deliver PAH to result in a reduction of plasma phenylalanine levels by 25% or greater. In another embodiment, the desirable endpoint is reducing plasma Phe levels to take subject to a “moderate” phenotype from a “severe” phenotype. Methods for measurement of phenylalanine levels are known in the art e.g, as described in Gregory et al, Blood phenylalanine monitoring for dietary compliance among patients with phenylketonuria: comparison of methods, Genetics in Medicine (November 2007) 9, 761-765, which is incorporated herein by reference. In one embodiment, patients achieve desired circulating PAH levels after treatment with rAAV.hPAH, alone and/or combined with the use of adjunctive treatments.

### 5.3. Dosing & Route of Administration

In one embodiment, the rAAV.hPAH vector is delivered as a single dose per patient. In one embodiment, the subject is delivered the minimal effective dose (MED) (as determined by preclinical study described in the Examples herein). As used herein, MED refers to the rAAV.hPAH dose required to achieve PAH activity resulting in Phe levels between 120-360 μmol/L.

As is conventional, the vector titer is determined on the basis of the DNA content of the vector preparation. In one embodiment, quantitative PCR or optimized quantitative PCR as described in the Examples is used to determine the DNA content of the rAAV.hPAH vector preparations. In one embodiment, digital droplet PCR as described in the Examples is used to determine the DNA content of the rAAV.hPAH vector preparations. In one embodiment, the dosage is about 1×10¹¹ genome copies (GC)/kg body weight to about 1×10¹³ GC/kg, inclusive of endpoints. In one embodiment, the dosage is 5×10¹¹ GC/kg. In another embodiment, the dosage is 5×10¹² GC/kg. In specific embodiments, the dose of rAAV.hPAH administered to a patient is at least 5×10¹¹ GC/kg, 1×10¹² GC/kg, 1.5×10¹² GC/kg, 2.0×10¹² GC/kg, 2.5×10¹² GC/kg, 3.0×10¹² GC/kg, 3.5×10¹² GC/kg, 4.0×10¹² GC/kg, 4.5×10¹² GC/kg, 5.0×10¹² GC/kg, 5.5×10¹² GC/kg, 6.0×10¹² GC/kg, 6.5×10¹² GC/kg, 7.0×10¹² GC/kg, or 7.5×10¹² GC/kg. Also, the replication-defective virus compositions can be formulated in dosage units to contain an amount of replication-defective virus that is in the range of about 1.0×10⁹ GC to about 1.0×10¹⁵ GC. As used herein, the term “dosage” can refer to the total dosage delivered to the subject in the course of treatment, or the amount delivered in a single (of multiple) administration.

In one embodiment, the dosage is sufficient to decrease plasma Phe levels in the patient by 25% or more.

In some embodiments, rAAV.hPAH is administered in combination with one or more therapies for the treatment of PKU, such as a low Phe diet or administration of sapropterin dihydrochloride.

### 5.4. Measuring Clinical Objectives

Measurements of efficacy of treatment can be measured by transgene expression and activity as determined by plasma Phe levels and/or PAH activity. Further assessment of efficacy can be determined by clinical assessment of dietary Phe tolerance.

As used herein, the rAAV.hPAH vector herein “functionally replaces” or “functionally supplements” the patients defective PAH with active PAH when the patient expresses a sufficient level of PAH to achieve PAH activity resulting in Phe levels between 120-360 μmol/L.

The following examples are illustrative only and are not intended to limit the present invention.

### EXAMPLES

The following examples are illustrative only and are not intended to limit the present invention.

### Example 1: Mouse Models of Phenylketonuria (PKU)

PAH^{-/-} mice were generated by CRISPR/Cas9 technology at Jackson Labs. Wild-type C57BL/6 mice were injected with Cas9 mRNA and two guide RNAs (sgRNA) targeting the second coding exon of PAH gene directly into mouse zygotes. Mice that developed from these embryos were sequenced to determine the mutation(s) and then bred with C57BL/6J mice to transmit the allele and confirm germline transmission.

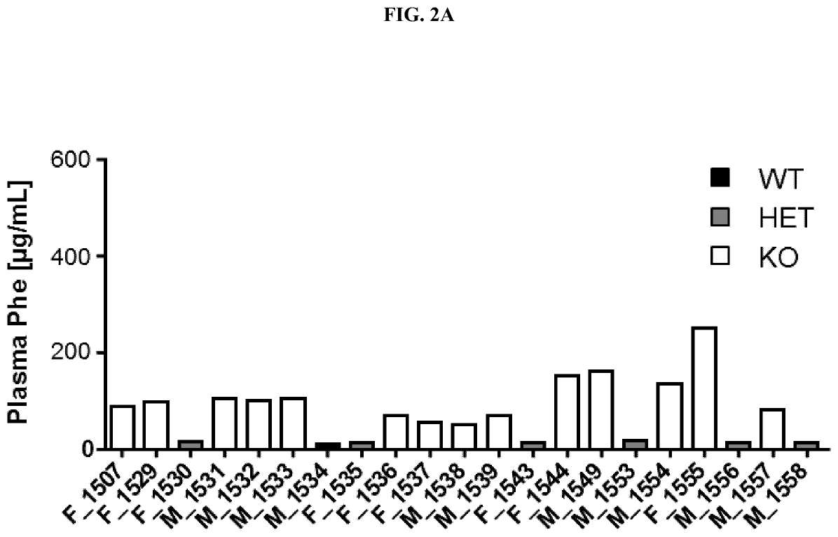

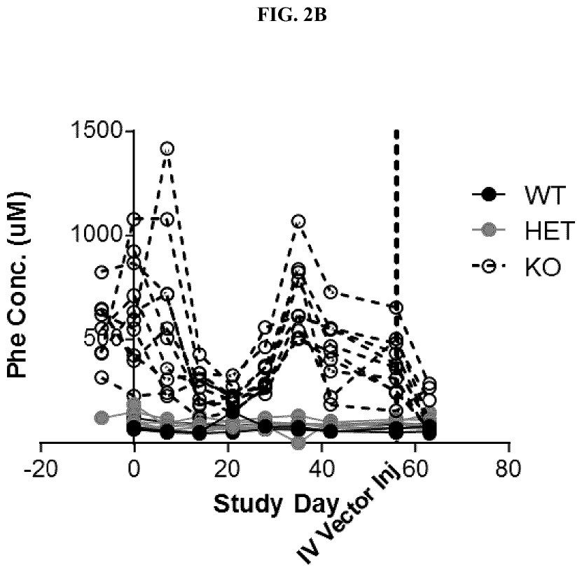

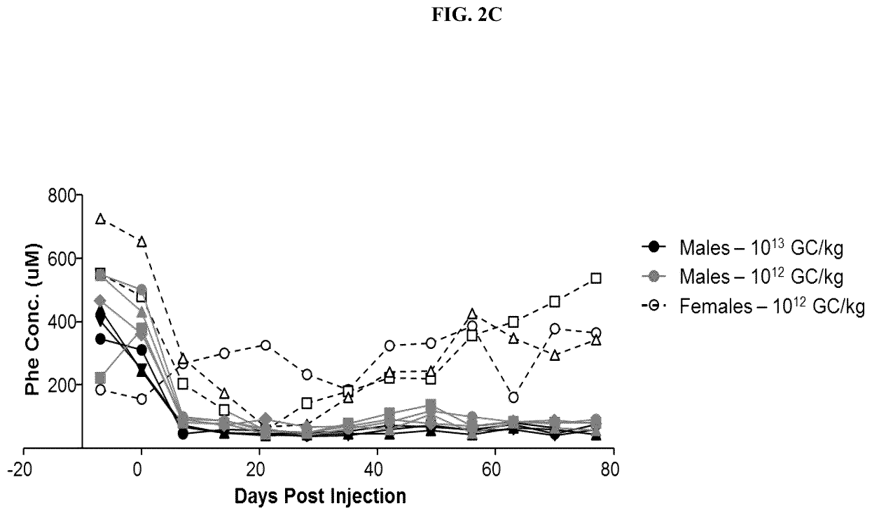

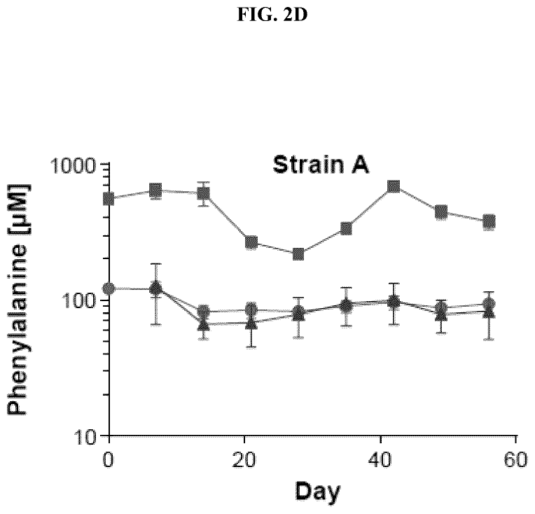

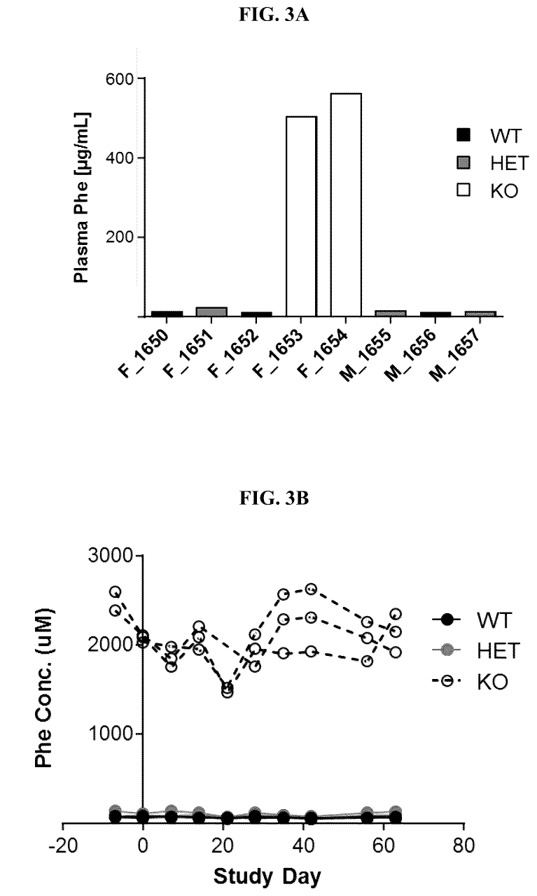

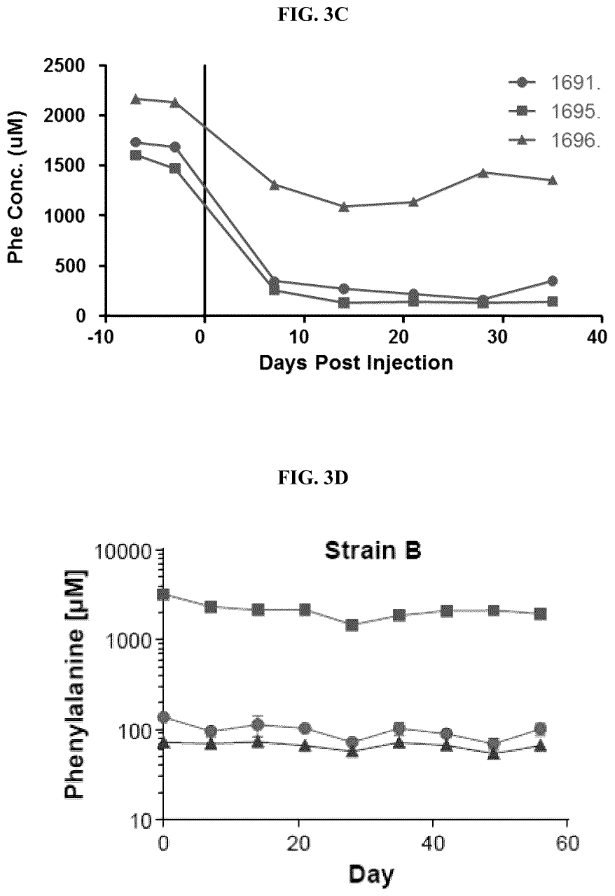

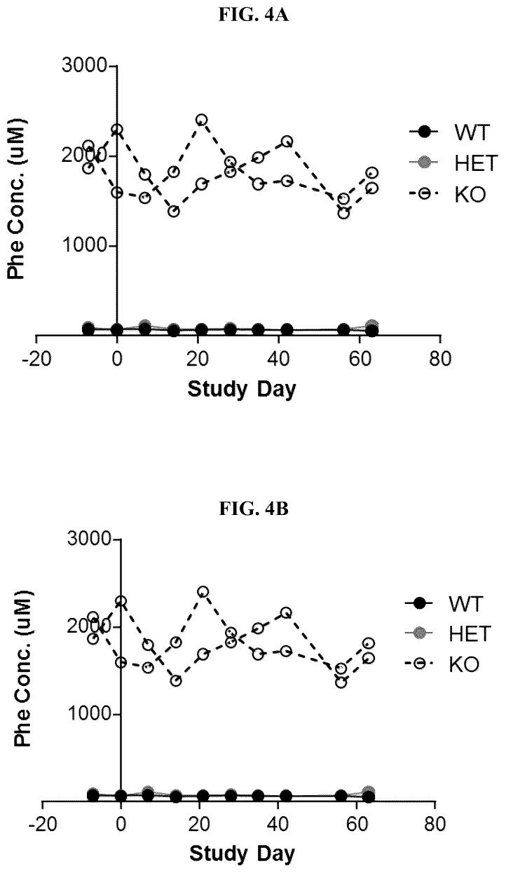

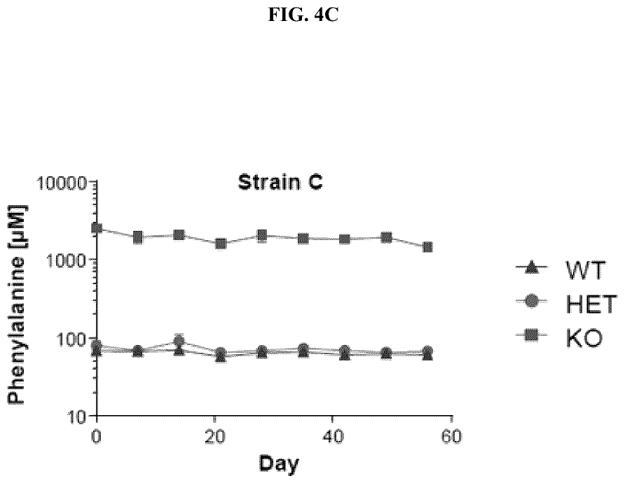

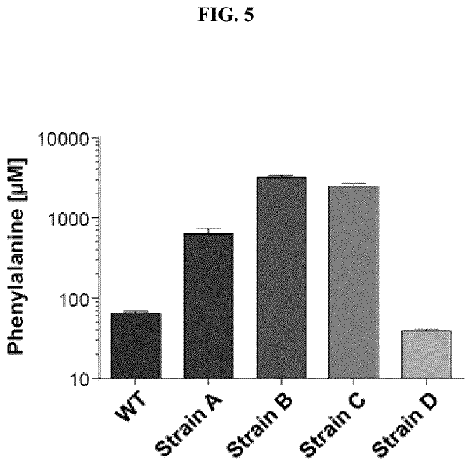

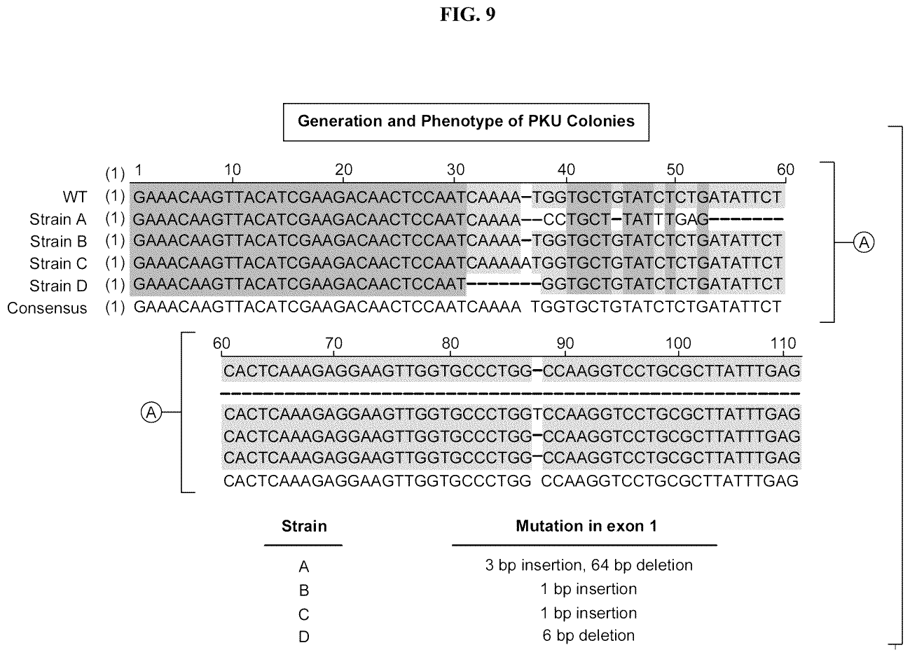

Four different mutations were generated and the mice strains were designated as PAH_KO_A, PAH_KO_B, PAH_KO_C, and PAH_KO_D. PAH_KO_A mice demonstrated a 3-bp substitution followed by a 64-bp deletion from the 6534 nt to the 6600 nt of Mus musculus PAH gene [NC_000076.6]. PAH_KO_B and PAH_KO_C mice showed a single base pair insertion after the 6589 nt and the 6539 nt respectively. PAH_KO_D showed a 6 bp deletion from the 6535 nt to the 6540 nt. FIG. 9 shows an alignment of a portion of the PAH sequence for wild type, PAH_KO_A, PAH_KO_B, PAH_KO_C, PAH_KO_D and consensus. A natural history study of these mice was performed. Blood samples were collected via retro orbital or submandibular bleeding. Plasma phenylalanine (Phe) levels were detected via LC/MS/MS and the data from PAH_KO_A, PAH_KO_B and PAH_KO_C mice was acquired and presented in FIGS. 2A, 3A and 4A respectively, and summarized in FIG. 5A. Heterozygous and wild-type littermates were provided as negative controls. Compared to the controls, the plasma phenylalanine levels in PAH_KO_A, PAH_KO_B and PAH_KO_C mice were significantly higher, indicating a functionally deficient PAH in these mice. It also suggested that PAH_KO_A, PAH_KO_B, and PAH_KO_C mice could serve as mouse models for Phenylketonuria (PKU) in human.

However, the fourth knock-out mice, PAH_KO_D, did not exhibit an elevated plasma phenylalanine level and thus excluded from further analysis.

### Example 2: AAV Vectors Containing hPAH-AAV8.TBG.PI.hPAHco.WPRE.bGH

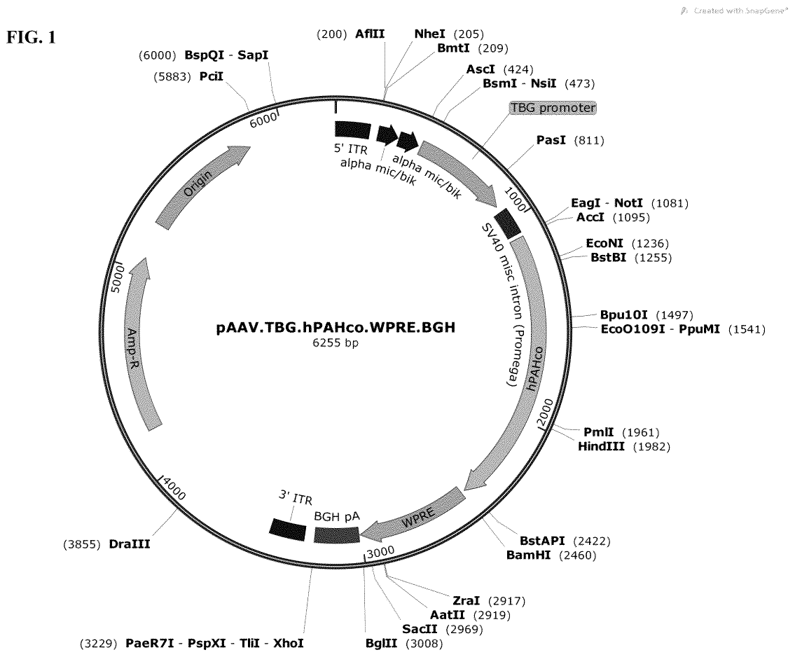

The gene therapy vector AAV8.TBG.PI.hPAHco. WPRE.bGH was constructed by an AAV8 vector bearing a codon-optimized human PAH cDNA under the control of TBG, a hybrid promoter based on the human thyroid hormone-binding globulin promoter and microglobin/bikunin enhancer (FIG. 1). The PAH expression cassette was flanked by AAV2 derived inverted terminal repeats (ITRs) and the expression was driven by a hybrid of the TBG enhancer/promoter and the Woodchuck Hepatitis Virus (WHP) posttranscriptional regulatory element (WPRE) as an enhancer. The transgene also included the Promega SV40 misc intron (PI) and a bovine growth hormone polyadenylation signal (bGH). The vector genome sequence is shown in SEQ ID NO: 20.

The vector was prepared using conventional triple transfection techniques in 293 cells as described e.g., by Mizukami, Hiroaki, et al. A Protocol for AAV vector production and purification. Diss. Di-vision of Genetic Therapeutics, Center for MolecularMedicine, 1998, which is incorporated herein by reference. All vectors were produced by the Vector Core at the University of Pennsylvania as previously described [Lock, M., et al, Hum Gene Ther, 21:1259-1271 (2010)].

### Example 3: AAV8.hPAH Vectors in the Model of PKU

All animal procedures were performed in accordance with protocols approved by the Institutional Animal Care and Use Committee (IACUC) of the University of Pennsylvania.

Twenty PAH_KO_A mice were generated. Seven males and three females were assessed in natural history study. On Day 56 of natural history study, three male mice with Identification Number 1531, 1532 and 1533 were injected intravenously via the tail vein with 1×10¹³ GC/kg of the AAV8.TBG.PI.hPAHco. WPRE.bGH vector. Four male mice with Identification Number 1538, 1539, 1554 and 1564 were injected with 1×10¹² GC/kg of the vector. Three female mice with Identification Number 1507, 1536 and 1537 were injected with 1×10¹² GC/kg of the vector. The blood samples were collected weekly to evaluate plasma phenylalanine concentration (FIG. 2B). A higher level of phenylalanine was detected in the PAH_KO_A mice before injection compared to the littermate controls, indicating a deficient PAH in PAH_KO_A mice. 7 days after the vector injection, the plasma phenylalanine level of PAH_KO_A mice decreased while the controls remained stable. This result demonstrated that a single injection of AAV8.TBG.PI.hPAHco. WPRE.bGH into PAH_KO_A mice could rescue the deficient PAH and reduce the pathological accumulation of phenylalanine in the blood.

The effects of gender differences and the two doses of the AAV8.TBG.PI.hPAHco. WPRE.bGH vectors were further evaluated in PAH_KO_A mice (FIG. 2C). Plasma phenylalanine levels were observed every week for 11 weeks, e.g, as described in Gregory et al, Blood phenylalanine monitoring for dietary compliance among patients with phenylketonuria: comparison of methods, Genetics in Medicine (November 2007) 9, 761-765, which is incorporated herein by reference. Two of three female mice received 1×10¹² GC/kg of the vectors and all seven male ones with both doses at 1×10¹² GC/kg and 1×10¹³ GC/kg displayed a reduced phenylalanine concentration in the plasma. The phenylalanine of the seven male mice maintained at comparably low levels for the 11-week observation period while all three female ones demonstrated a slow increase in plasma phenylalanine level.

Three female PAH_KO_B mice were generated and exanimated in a natural history study. Weekly bleeds for phenylalanine levels were performed and the result confirmed an abnormal accumulation of phenylalanine in the blood compared to the healthy littermate controls. Upon intravenous injection of 1×10¹² GC/kg of AAV8.TBG.PI.hPAHco. WPRE.bGH, a decreased phenylalanine level was observed in all three females and the low level maintained during the 8-week observation period after the injection.

Two male PAH_KO_C mice were generated for this study and utilized in natural history study. A weekly collection of blood samples were performed and the phenylalanine concentration was assessed. The data showed that during the 9-week observation, both PAH_KO_C mice and the heterozygous/wild-type littermates maintained a comparably stable concentration of plasma phenylalanine while the knock-out mice demonstrated a significantly higher level.

A further study of expression and enzyme activity of PAH in the injected PAH^{-/-} mice was performed. Livers are collected from PAH_KO_A, PAH_KO_B and PAH_KO_C mice injected with vectors or PBS only as well as the healthy littermate controls. mRNA is extracted from the liver and the expression of human PAH is evaluated via RT-PCR. To determine the protein expression of PAH, liver lysates are prepared for detection by western blot while liver sections are prepared for immunohistochemistry. Experiments are also performed to assess the PAH enzyme activity of the PAH/mice treated with the vector as well as controls.

To fully evaluate gender difference in all three PAH-mice, PAH_KO_A, PAH_KO_B and PAH_KO_C mice were bred to assess the plasma phenylalanine concentration, the expression of PAH on both mRNA and protein level and the enzyme activity of PAH before and after the injection of AAV8.TBG.PI.hPAHco. WPRE.bGH.

To determine the dose-dependent expression of AAV8.TBG.PI.hPAHco. WPRE.bGH and the potential toxicity of the highest dose, various doses of AAV8.TBG.PI.hPAHco. WPRE.bGH are injected into the PAH^{-/-} mice and further assessment of phenylalanine accumulation and PAH expression/activity are performed.

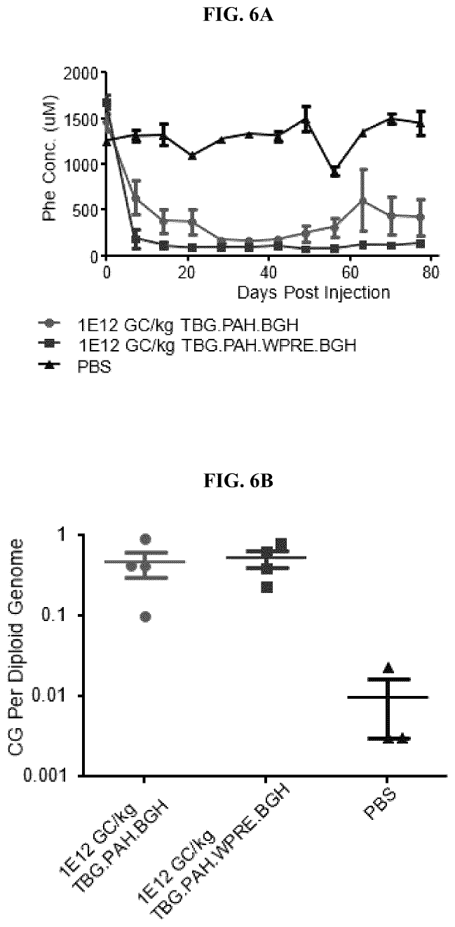

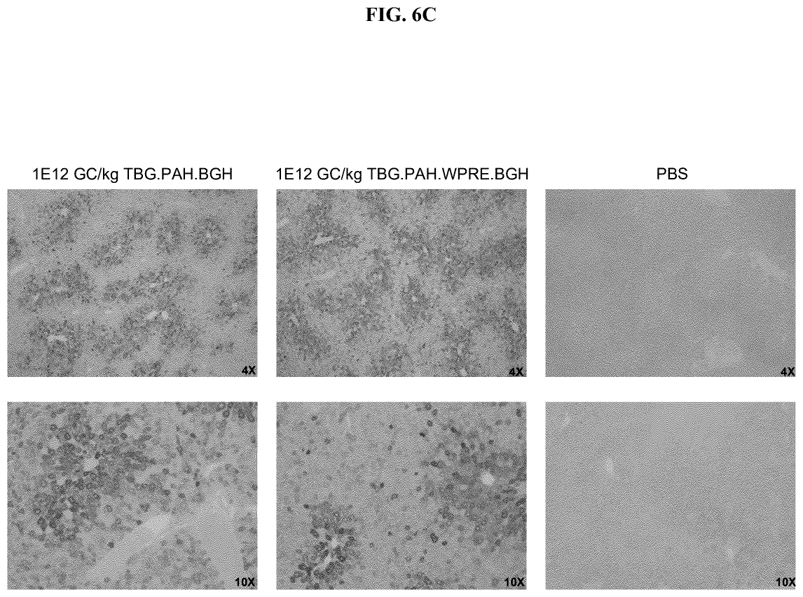

Similar experiments were performed with AAV8.TBG.hPAHco.bGH. PKU B mice ages 17-22 weeks were given either 10¹² GC/kg of either AAV8.TBG.hPAHco.bGH or AAV8.TBG.hPAHco. WPRE.bGH (or PBS for control) after pretreatment phenylalanine levels were established. Mice were then bled weekly, and plasma was isolated and phenylalanine concentration was determined (FIG. 6A). At the termination of the study, liver was collected and genome copy analysis (FIG. 6B) and immunohistochemistry (FIG. 6C) was performed. Phenylalanine levels were reduced in both AAV8.TBG.hPAHco.bGH and AAV8.TBG.hPAHco. WPRE.bGH treated mice.

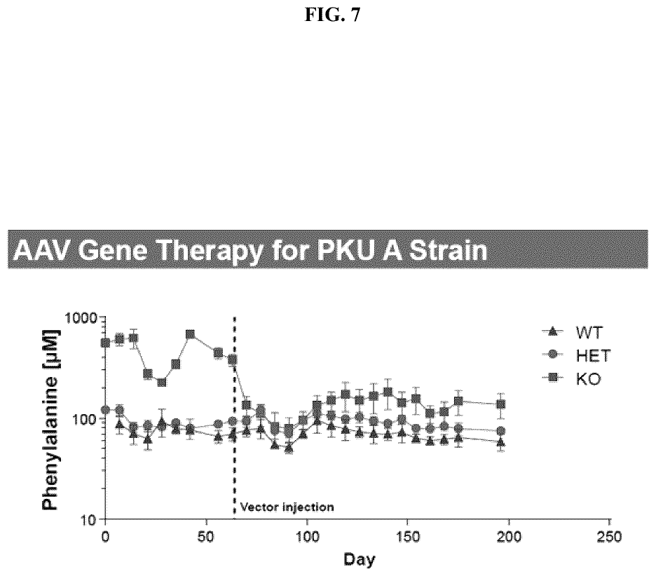

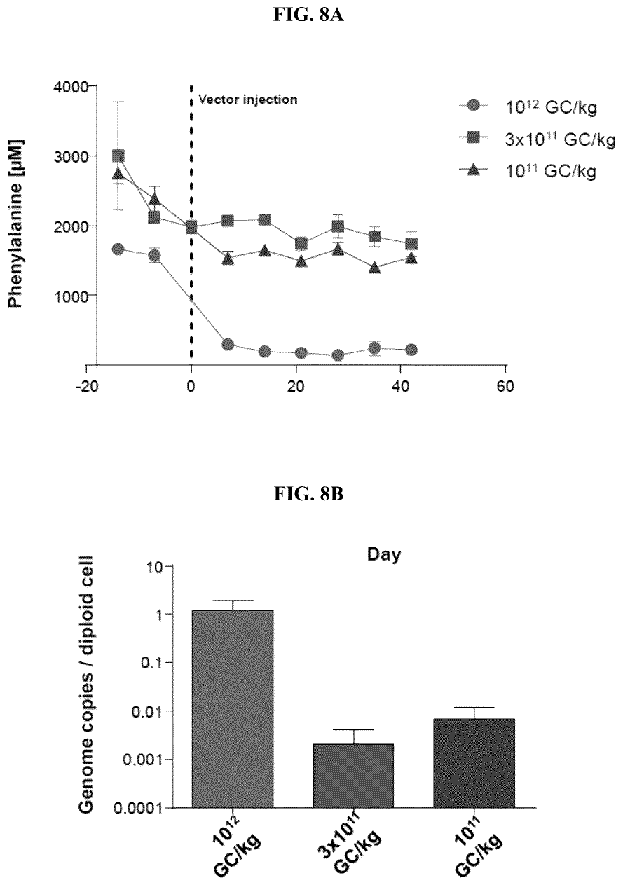

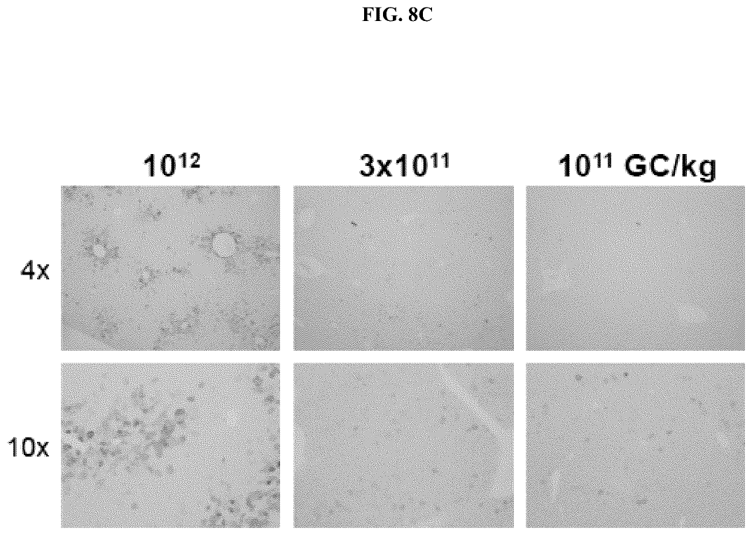

Further studies were performed with AAV8.TBG.hPAHco.bGH vector. Wildtype, heterozygous (circle) and PKU_KO_A (KO) mice were injected intravenously with 10¹¹ GC/kg of AAV8.TBG.hPAHco after baseline phenylalanine levels were established. Mice were then bled weekly, and plasma was isolated and analyzed for Phe concentration. Phe levels in plasma decreased by 71% following intravenous administration of AAV8.TBG.hPAHco. FIG. 7.

PKU_KO_B mice were given either 10¹² GC/kg, 3×10¹¹ GC/kg, or 10¹¹ GC/kg, of AAV8.TBG.hPAHco after baseline phenylalanine levels were established. Mice were then bled weekly, and plasma was isolated and analyzed for Phe concentration. At termination of the study, liver was collected and genome copy analysis and immunohistochemistry was performed. Protein expression and reduction in Phe levels were seen at a dose of 10¹² GC/kg.

Meanwhile, administration of 10¹² GC/kg of each of the following vectors, AAV8.TBG.PI.hPAHco.bGH, AAV8.LSP.IVS2.hPAHco.bGH, AAV8.A1AT.hPAHco.BGH, AAV8.TTR.hPAHco.BGH, AAV8.TBG.PI.hPAHnativesequence.bGH, AAV8.ABPS.TBG.hFIXintron.hPAHco.BGH, AAV8.ABPS.TBG-S1.hFIXintron.hPAHco.BGH, AAV8.ApoE.A1AT.hFIXintron.hPAHco.BGH, is also performed and served as a comparison.

In conclusion, a single injection of AAV8.TBG.PI.hPAHco. WPRE.bGH vector resulted in substantial plasma phenylalanine reduction and concomitant functional correction when administered intravenously in three PAH-deficient mice.

### Example 4: AAV Gene Therapy for Phenylketonuria

Phenylketonuria (PKU) is an autosomal recessive genetic disorder caused by the attenuation of phenylalanine-4-hydroxylase (PAH) activity, resulting in the buildup of phenylalanine in the tissues and blood. High levels of phenylalanine in the bloodstream are thought to inhibit the transport of other large neutral amino acids across the blood brain barrier, affecting brain development and resulting in intellectual disability and seizures. Treatment for PKU is currently limited to maintenance of a strict phenylalanine-restricted diet and products directed at stabilizing residual PAH. A liver-targeted AAV gene therapy approach described herein is to improve upon the current standard of care.

To investigate the development of gene therapy for PKU, four unique mouse strains were created by inducing different mutations in exon 1 of the PAH gene by CRISPR/Cas9 technology as described herein. A natural history study was performed on each of these strains to determine the progression of the disease and identify the strain that best replicated the human PKU phenotype. PKU colonies, designated B and C, both contained a single base pair (bp) deletion at different locations in exon 1 and maintained average phenylalanine levels of 2049 μM and 1705 μM, respectively, compared to normal levels of 70 μM. PKU colony A, despite having a 64 bp deletion and a 3 bp insertion in exon 1 of the PAH gene, had a modestly higher average phenylalanine level of 477 μM. PKU colony D, which had a 6 bp deletion, had phenylalanine levels equivalent to wild type littermates. Following AAV8 vector administration at a dose of 1×10¹² GC/kg for expression of a human codon optimized version of PAH to the PKU B mouse colony, plasma phenylalanine levels were reduced by 87% to 222 μM. This reduction in plasma phenylalanine levels restored the ability of the males to produce offspring. These results represent development of an AAV-based therapeutic for PKU.

All publications cited in this specification are incorporated herein by reference. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (AAV) comprising an AAV capsid and a vector genome packaged therein, said vector genome comprising:
a. an AAV 5′ inverted terminal repeat (ITR) sequence;
b. two copies of an α1-microglogubulin/bikunin precursor enhancer sequence;
c. a promoter sequence;
d. a nucleic acid sequence encoding a human phenylalanine hydroxylase (PAH); and
e. an AAV 3′ ITR sequence;
wherein the nucleic acid sequence encoding human PAH is operably linked to two copies of the α1-microglogubulin/bikunin precursor enhancer sequence.

2. The recombinant AAV of claim 1, wherein the AAV capsid is an AAV8 capsid.

3. The recombinant AAV of claim 1, wherein the promoter sequence is selected from the group consisting of a thyroxine binding globulin (TBG) promoter sequence, a modified thyroxine binding globulin (TBG-S1) promoter sequence, an alpha-trypsin (A1AT) promoter sequence, a liver-specific promoter (LSP) promoter sequence, and a transthyretin (TTR) promoter sequence.

4. The recombinant AAV of claim 1, wherein the promoter sequence is an A1AT promoter sequence.

5. The recombinant AAV of claim 1, wherein the promoter sequence is an LSP promoter sequence.

6. The recombinant AAV of claim 1, wherein the promoter sequence is a transthyretin (TTR) promoter sequence.

7. The recombinant AAV of claim 1, wherein the AAV 5′ ITR sequence and/or the AAV3′ ITR sequence is from an AAV2 sequence.

8. The recombinant AAV of claim 1, wherein the vector genome further comprises a polyA sequence.

9. The recombinant AAV of claim 8, wherein the polyA sequence is a bovine growth hormone (bGH) polyA sequence.

10. The recombinant AAV of claim 1, wherein the vector genome further comprises a woodchuck hepatitis virus post-transcriptional regulatory element (WPRE).

11. The recombinant AAV of claim 1, wherein the vector genome further comprises an intron.

12. The recombinant AAV of claim 11, wherein the intron is selected from the group consisting of human beta globin IVS2 and human beta globin SV40.

13. The recombinant AAV of claim 1, wherein the vector genome is about 3 kilobases to about 5.5 kilobases in length.

14. The recombinant AAV of claim 1, wherein the nucleic acid sequence encoding a human PAH comprises SEQ ID NO: 3, or a sequence at least 70% identical thereto.

15. The recombinant AAV of claim 1, wherein the nucleic acid sequence encoding a human PAH is codon optimized nucleic acid sequence encoding a human PAH.

16. An aqueous suspension suitable for administration to a phenylketonuria patient, said suspension comprising an aqueous suspending liquid and about 1×10¹² genome copies (GC)/mL to about 1×10¹⁴ GC/mL of a recombinant adeno-associated virus (AAV) useful as a liver-directed therapeutic for phenylketonuria, said recombinant AAV having an AAV capsid, and having packaged therein a vector genome comprising:
a. an AAV 5′ inverted terminal repeat (ITR) sequence;
b. two copies of an al-microglogubulin/bikunin precursor enhancer sequence;
c. a promoter sequence;
d. a nucleic acid sequence encoding a human phenylalanine hydroxylase (PAH); and
e. an AAV 3′ ITR sequence;
wherein the nucleic acid sequence encoding a human PAH is operably linked to two copies of the α1-microglogubulin/bikunin precursor enhancer sequence.

17. The aqueous suspension suitable for administration to a phenylketonuria patient of claim 16, wherein the nucleic acid sequence encoding a human PAH comprises SEQ ID NO: 3, or a sequence at least 70% identical thereto.

18. The aqueous suspension suitable for administration to a phenylketonuria patient of claim 16, wherein the nucleic acid sequence encoding a human PAH is codon optimized nucleic acid sequence encoding a human PAH.

19. The aqueous suspension suitable for administration to a phenylketonuria patient of claim 16, wherein the recombinant AAV capsid is an AAV8 capsid.

20. The aqueous suspension suitable for administration to a phenylketonuria patient of claim 16, wherein the suspension is suitable for intravenous injection.

21. The aqueous suspension suitable for administration to a phenylketonuria patient of claim 16, wherein the suspension further comprises a surfactant, preservative, and/or buffer dissolved in the aqueous suspending liquid.

22. A method of treating a patient having phenylketonuria with a recombinant adeno-associated virus (AAV) according to claim 1, wherein the recombinant AAV is delivered at about 1×10¹⁰ genome copies (GC)/kg to about 1×10¹⁵ GC/kg in an aqueous suspension, wherein the GC are calculated as determined based on oqPCR or ddPCR.

23. A plasmid comprising an expression cassette, the expression cassette comprising two copies of an al-microglogubulin/bikunin precursor enhancer sequence, a TBG promoter sequence, a nucleic acid sequence encoding a human phenylalanine hydroxylase (PAH), and a bGH polyA sequence, wherein the TBG promoter sequence, codon optimized sequence encoding a human phenylalanine hydroxylase (PAH), and bGH polyA sequence are operably linked.

24. The plasmid of claim 23, wherein the TBG promoter sequence comprises SEQ ID NO: 9 and/or the bGH polyA sequence comprises SEQ ID NO: 12.

25. The plasmid of claim 23, wherein the expression cassette is flanked by an AAV 5′ ITR sequence from AAV2 and/or an AAV 3′ ITR from AAV2.

26. The plasmid of claim 23, wherein the AAV 5′ ITR sequence from AAV2 comprises SEQ ID NO: 16 and/or the AAV 3′ ITR from AAV2 comprises SEQ ID NO: 17.

27. The plasmid of claim 23, wherein the nucleic acid sequence encoding a human PAH comprises SEQ ID NO: 3, or a sequence at least 70% identical thereto.

28. The plasmid of claim 23, wherein the nucleic acid sequence encoding a human PAH, is a codon optimized nucleic acid sequence encoding a human PAH.


SEQUENCE LISTING (31 sequences)

SEQ ID NO: 1 - DNA, 1356 bp, synthetic construct
  misc_feature 1..1356: note = constructed sequence
  source 1..1356: mol_type = other DNA
atgtctaccgccgtgctggaaaatcccggcctgggcagaaagctgagcgacttcggccaggaaaccagctacatcgaggacaactgcaaccagaacggcgccatcagcctgatcttcagcctgaaagaagaagtgggcgccctggccaaggtgctgcggctgttcgaagagaacgacgtgaacctgacccacatcgagagccggcccagcagactgaagaaggacgagtacgagttcttcacccacctggacaagcggagcctgcccgccctgaccaacatcatcaagatcctgcggcacgacatcggcgccaccgtgcacgagctgagccgggacaagaaaaaggacaccgtgccctggttcccccggaccatccaggaactggacagattcgccaaccagatcctgagctacggcgccgagctggacgccgatcaccccggctttaaggaccccgtgtaccgggccagacggaagcagtttgccgatatcgcctacaactaccggcacggccagcccatcccccgggtggagtatatggaagaggaaaagaaaacctggggcaccgtgttcaagaccctgaagtccctgtacaagacccacgcctgctacgagtacaaccacatcttcccactgctggaaaagtactgcggcttccacgaggacaatatcccccagctggaagacgtgtcccagttcctgcagacctgcaccggcttcagactgaggcctgtggccggactgctgagcagcagagattttctgggcggactggccttccgggtgttccactgcacccagtacatcagacacggcagcaagcccatgtacacccccgagcccgatatctgccacgagctgctgggacacgtgcccctgttcagcgacagaagcttcgcccagttcagccaggaaatcggcctggcctctctgggagcccccgacgagtatatcgagaagctggccaccatctactggttcaccgtggaattcggcctgtgcaagcagggcgacagcatcaaggcctacggcgctggcctgctgtccagctttggcgagctgcagtactgtctgagcgagaagcccaagctgctgcccctggaactggaaaagaccgccatccagaactacaccgtgaccgagttccagcccctgtactacgtggccgagagcttcaacgacgccaaagaaaaagtgcggaacttcgccgccaccatccccagaccattctccgtcagatacgacccctacacccagcggatcgaggtgctggacaacacacagcagctgaaaattctggccgactccatcaacagcgagatcggcatcctgtgcagcgccctgcagaagatcaag

SEQ ID NO: 2 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 3 - DNA, 1359 bp, Homo sapiens
  source 1..1359: mol_type = other DNA
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaa

SEQ ID NO: 4 - DNA, 34 bp, synthetic construct
  misc_feature 1..34: note = constructed sequence
  source 1..34: mol_type = other DNA
tgtttgctgcttgcaatgtttgcccattttaggg

SEQ ID NO: 5 - DNA, 100 bp, synthetic construct
  misc_feature 1..100: note = constructed sequence
  source 1..100: mol_type = other DNA
ctacctcgtgatcgcccggcccctgttcaaacatgtcctaatactctgtctctgcaagggtcatcagtagttttccatcttactcaacatcctcccagtg

SEQ ID NO: 6 - DNA, 42 bp, synthetic construct
  misc_feature 1..42: note = constructed sequence
  source 1..42: mol_type = other DNA
aggttaatttttaaactgtttgctctggttaataatctcagg

SEQ ID NO: 7 - DNA, 322 bp, synthetic construct
  misc_feature 1..322: note = constructed sequence
  source 1..322: mol_type = other DNA
aaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 8 - DNA, 176 bp, synthetic construct
  misc_feature 1..176: note = constructed sequence
  source 1..176: mol_type = other DNA
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 9 - DNA, 477 bp, synthetic construct
  misc_feature 1..477: note = constructed sequence
  source 1..477: mol_type = other DNA
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 10 - DNA, 218 bp, synthetic construct
  misc_feature 1..218: note = constructed sequence
  source 1..218: mol_type = other DNA
tggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaata

SEQ ID NO: 11 - DNA, 190 bp, synthetic construct
  misc_feature 1..190: note = constructed sequence
  source 1..190: mol_type = other DNA
atttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 12 - DNA, 215 bp, synthetic construct
  misc_feature 1..215: note = constructed sequence
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 13 - DNA, 572 bp, synthetic construct
  misc_feature 1..572: note = constructed sequence
  source 1..572: mol_type = other DNA
agcttacttgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattg

SEQ ID NO: 14 - DNA, 1356 bp, synthetic construct; identical to SEQ ID NO: 1
  misc_feature 1..1356: note = constructed sequence
  source 1..1356: mol_type = other DNA

SEQ ID NO: 15 - DNA, 542 bp, synthetic construct
  misc_feature 1..542: note = constructed sequence
  source 1..542: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 16 - DNA, 168 bp, synthetic construct
  misc_feature 1..168: note = constructed sequence
  source 1..168: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 17 - DNA, 168 bp, synthetic construct
  misc_feature 1..168: note = constructed sequence
  source 1..168: mol_type = other DNA
cgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 1421 bp, synthetic construct
  misc_feature 1..1421: note = constructed sequence
  source 1..1421: mol_type = other DNA
gtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaaagcttttcatggattaggaaaaaatcattttgtctctatgtcaaacatcttggagttgatatttggggaaacacaatactcagttgagttccctaggggagaaaagcaagcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcag

SEQ ID NO: 19 - protein, 738 aa, unidentified
  REGION 1..738: note = AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 20 - DNA, 3445 bp, synthetic construct
  misc_feature 1..3445: note = constructed sequence
  source 1..3445: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgtctaccgccgtgctggaaaatcccggcctgggcagaaagctgagcgacttcggccaggaaaccagctacatcgaggacaactgcaaccagaacggcgccatcagcctgatcttcagcctgaaagaagaagtgggcgccctggccaaggtgctgcggctgttcgaagagaacgacgtgaacctgacccacatcgagagccggcccagcagactgaagaaggacgagtacgagttcttcacccacctggacaagcggagcctgcccgccctgaccaacatcatcaagatcctgcggcacgacatcggcgccaccgtgcacgagctgagccgggacaagaaaaaggacaccgtgccctggttcccccggaccatccaggaactggacagattcgccaaccagatcctgagctacggcgccgagctggacgccgatcaccccggctttaaggaccccgtgtaccgggccagacggaagcagtttgccgatatcgcctacaactaccggcacggccagcccatcccccgggtggagtatatggaagaggaaaagaaaacctggggcaccgtgttcaagaccctgaagtccctgtacaagacccacgcctgctacgagtacaaccacatcttcccactgctggaaaagtactgcggcttccacgaggacaatatcccccagctggaagacgtgtcccagttcctgcagacctgcaccggcttcagactgaggcctgtggccggactgctgagcagcagagattttctgggcggactggccttccgggtgttccactgcacccagtacatcagacacggcagcaagcccatgtacacccccgagcccgatatctgccacgagctgctgggacacgtgcccctgttcagcgacagaagcttcgcccagttcagccaggaaatcggcctggcctctctgggagcccccgacgagtatatcgagaagctggccaccatctactggttcaccgtggaattcggcctgtgcaagcagggcgacagcatcaaggcctacggcgctggcctgctgtccagctttggcgagctgcagtactgtctgagcgagaagcccaagctgctgcccctggaactggaaaagaccgccatccagaactacaccgtgaccgagttccagcccctgtactacgtggccgagagcttcaacgacgccaaagaaaaagtgcggaacttcgccgccaccatccccagaccattctccgtcagatacgacccctacacccagcggatcgaggtgctggacaacacacagcagctgaaaattctggccgactccatcaacagcgagatcggcatcctgtgcagcgccctgcagaagatcaagtgataagcatgcggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 2897 bp, synthetic construct
  misc_feature 1..2897: note = constructed sequence
  source 1..2897: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgtctaccgccgtgctggaaaatcccggcctgggcagaaagctgagcgacttcggccaggaaaccagctacatcgaggacaactgcaaccagaacggcgccatcagcctgatcttcagcctgaaagaagaagtgggcgccctggccaaggtgctgcggctgttcgaagagaacgacgtgaacctgacccacatcgagagccggcccagcagactgaagaaggacgagtacgagttcttcacccacctggacaagcggagcctgcccgccctgaccaacatcatcaagatcctgcggcacgacatcggcgccaccgtgcacgagctgagccgggacaagaaaaaggacaccgtgccctggttcccccggaccatccaggaactggacagattcgccaaccagatcctgagctacggcgccgagctggacgccgatcaccccggctttaaggaccccgtgtaccgggccagacggaagcagtttgccgatatcgcctacaactaccggcacggccagcccatcccccgggtggagtatatggaagaggaaaagaaaacctggggcaccgtgttcaagaccctgaagtccctgtacaagacccacgcctgctacgagtacaaccacatcttcccactgctggaaaagtactgcggcttccacgaggacaatatcccccagctggaagacgtgtcccagttcctgcagacctgcaccggcttcagactgaggcctgtggccggactgctgagcagcagagattttctgggcggactggccttccgggtgttccactgcacccagtacatcagacacggcagcaagcccatgtacacccccgagcccgatatctgccacgagctgctgggacacgtgcccctgttcagcgacagaagcttcgcccagttcagccaggaaatcggcctggcctctctgggagcccccgacgagtatatcgagaagctggccaccatctactggttcaccgtggaattcggcctgtgcaagcagggcgacagcatcaaggcctacggcgctggcctgctgtccagctttggcgagctgcagtactgtctgagcgagaagcccaagctgctgcccctggaactggaaaagaccgccatccagaactacaccgtgaccgagttccagcccctgtactacgtggccgagagcttcaacgacgccaaagaaaaagtgcggaacttcgccgccaccatccccagaccattctccgtcagatacgacccctacacccagcggatcgaggtgctggacaacacacagcagctgaaaattctggccgactccatcaacagcgagatcggcatcctgtgcagcgccctgcagaagatcaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 3341 bp, synthetic construct
  misc_feature 1..3341: note = constructed sequence
  source 1..3341: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcagcttacttgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgaggccgccaccatgtctaccgccgtgctggaaaatcccggcctgggcagaaagctgagcgacttcggccaggaaaccagctacatcgaggacaactgcaaccagaacggcgccatcagcctgatcttcagcctgaaagaagaagtgggcgccctggccaaggtgctgcggctgttcgaagagaacgacgtgaacctgacccacatcgagagccggcccagcagactgaagaaggacgagtacgagttcttcacccacctggacaagcggagcctgcccgccctgaccaacatcatcaagatcctgcggcacgacatcggcgccaccgtgcacgagctgagccgggacaagaaaaaggacaccgtgccctggttcccccggaccatccaggaactggacagattcgccaaccagatcctgagctacggcgccgagctggacgccgatcaccccggctttaaggaccccgtgtaccgggccagacggaagcagtttgccgatatcgcctacaactaccggcacggccagcccatcccccgggtggagtatatggaagaggaaaagaaaacctggggcaccgtgttcaagaccctgaagtccctgtacaagacccacgcctgctacgagtacaaccacatcttcccactgctggaaaagtactgcggcttccacgaggacaatatcccccagctggaagacgtgtcccagttcctgcagacctgcaccggcttcagactgaggcctgtggccggactgctgagcagcagagattttctgggcggactggccttccgggtgttccactgcacccagtacatcagacacggcagcaagcccatgtacacccccgagcccgatatctgccacgagctgctgggacacgtgcccctgttcagcgacagaagcttcgcccagttcagccaggaaatcggcctggcctctctgggagcccccgacgagtatatcgagaagctggccaccatctactggttcaccgtggaattcggcctgtgcaagcagggcgacagcatcaaggcctacggcgctggcctgctgtccagctttggcgagctgcagtactgtctgagcgagaagcccaagctgctgcccctggaactggaaaagaccgccatccagaactacaccgtgaccgagttccagcccctgtactacgtggccgagagcttcaacgacgccaaagaaaaagtgcggaacttcgccgccaccatccccagaccattctccgtcagatacgacccctacacccagcggatcgaggtgctggacaacacacagcagctgaaaattctggccgactccatcaacagcgagatcggcatcctgtgcagcgccctgcagaagatcaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 2203 bp, synthetic construct
  misc_feature 1..2203: note = constructed sequence
  source 1..2203: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattctggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagcggccgccaccatgtctaccgccgtgctggaaaatcccggcctgggcagaaagctgagcgacttcggccaggaaaccagctacatcgaggacaactgcaaccagaacggcgccatcagcctgatcttcagcctgaaagaagaagtgggcgccctggccaaggtgctgcggctgttcgaagagaacgacgtgaacctgacccacatcgagagccggcccagcagactgaagaaggacgagtacgagttcttcacccacctggacaagcggagcctgcccgccctgaccaacatcatcaagatcctgcggcacgacatcggcgccaccgtgcacgagctgagccgggacaagaaaaaggacaccgtgccctggttcccccggaccatccaggaactggacagattcgccaaccagatcctgagctacggcgccgagctggacgccgatcaccccggctttaaggaccccgtgtaccgggccagacggaagcagtttgccgatatcgcctacaactaccggcacggccagcccatcccccgggtggagtatatggaagaggaaaagaaaacctggggcaccgtgttcaagaccctgaagtccctgtacaagacccacgcctgctacgagtacaaccacatcttcccactgctggaaaagtactgcggcttccacgaggacaatatcccccagctggaagacgtgtcccagttcctgcagacctgcaccggcttcagactgaggcctgtggccggactgctgagcagcagagattttctgggcggactggccttccgggtgttccactgcacccagtacatcagacacggcagcaagcccatgtacacccccgagcccgatatctgccacgagctgctgggacacgtgcccctgttcagcgacagaagcttcgcccagttcagccaggaaatcggcctggcctctctgggagcccccgacgagtatatcgagaagctggccaccatctactggttcaccgtggaattcggcctgtgcaagcagggcgacagcatcaaggcctacggcgctggcctgctgtccagctttggcgagctgcagtactgtctgagcgagaagcccaagctgctgcccctggaactggaaaagaccgccatccagaactacaccgtgaccgagttccagcccctgtactacgtggccgagagcttcaacgacgccaaagaaaaagtgcggaacttcgccgccaccatccccagaccattctccgtcagatacgacccctacacccagcggatcgaggtgctggacaacacacagcagctgaaaattctggccgactccatcaacagcgagatcggcatcctgtgcagcgccctgcagaagatcaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 2496 bp, synthetic construct
  misc_feature 1..2496: note = constructed sequence
  source 1..2496: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcctacctcgtgatcgcccggcccctgttcaaacatgtcctaatactctgtctctgcaagggtcatcagtagttttccatcttactcaacatcctcccagtggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgtctaccgccgtgctggaaaatcccggcctgggcagaaagctgagcgacttcggccaggaaaccagctacatcgaggacaactgcaaccagaacggcgccatcagcctgatcttcagcctgaaagaagaagtgggcgccctggccaaggtgctgcggctgttcgaagagaacgacgtgaacctgacccacatcgagagccggcccagcagactgaagaaggacgagtacgagttcttcacccacctggacaagcggagcctgcccgccctgaccaacatcatcaagatcctgcggcacgacatcggcgccaccgtgcacgagctgagccgggacaagaaaaaggacaccgtgccctggttcccccggaccatccaggaactggacagattcgccaaccagatcctgagctacggcgccgagctggacgccgatcaccccggctttaaggaccccgtgtaccgggccagacggaagcagtttgccgatatcgcctacaactaccggcacggccagcccatcccccgggtggagtatatggaagaggaaaagaaaacctggggcaccgtgttcaagaccctgaagtccctgtacaagacccacgcctgctacgagtacaaccacatcttcccactgctggaaaagtactgcggcttccacgaggacaatatcccccagctggaagacgtgtcccagttcctgcagacctgcaccggcttcagactgaggcctgtggccggactgctgagcagcagagattttctgggcggactggccttccgggtgttccactgcacccagtacatcagacacggcagcaagcccatgtacacccccgagcccgatatctgccacgagctgctgggacacgtgcccctgttcagcgacagaagcttcgcccagttcagccaggaaatcggcctggcctctctgggagcccccgacgagtatatcgagaagctggccaccatctactggttcaccgtggaattcggcctgtgcaagcagggcgacagcatcaaggcctacggcgctggcctgctgtccagctttggcgagctgcagtactgtctgagcgagaagcccaagctgctgcccctggaactggaaaagaccgccatccagaactacaccgtgaccgagttccagcccctgtactacgtggccgagagcttcaacgacgccaaagaaaaagtgcggaacttcgccgccaccatccccagaccattctccgtcagatacgacccctacacccagcggatcgaggtgctggacaacacacagcagctgaaaattctggccgactccatcaacagcgagatcggcatcctgtgcagcgccctgcagaagatcaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 2241 bp, synthetic construct
  misc_feature 1..2241: note = constructed sequence
  source 1..2241: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctacctcgtgatcgcccggcccctgttcaaacatgtcctaatactctgtctctgcaagggtcatcagtagttttccatcttactcaacatcctcccagtggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgccaccatgtctaccgccgtgctggaaaatcccggcctgggcagaaagctgagcgacttcggccaggaaaccagctacatcgaggacaactgcaaccagaacggcgccatcagcctgatcttcagcctgaaagaagaagtgggcgccctggccaaggtgctgcggctgttcgaagagaacgacgtgaacctgacccacatcgagagccggcccagcagactgaagaaggacgagtacgagttcttcacccacctggacaagcggagcctgcccgccctgaccaacatcatcaagatcctgcggcacgacatcggcgccaccgtgcacgagctgagccgggacaagaaaaaggacaccgtgccctggttcccccggaccatccaggaactggacagattcgccaaccagatcctgagctacggcgccgagctggacgccgatcaccccggctttaaggaccccgtgtaccgggccagacggaagcagtttgccgatatcgcctacaactaccggcacggccagcccatcccccgggtggagtatatggaagaggaaaagaaaacctggggcaccgtgttcaagaccctgaagtccctgtacaagacccacgcctgctacgagtacaaccacatcttcccactgctggaaaagtactgcggcttccacgaggacaatatcccccagctggaagacgtgtcccagttcctgcagacctgcaccggcttcagactgaggcctgtggccggactgctgagcagcagagattttctgggcggactggccttccgggtgttccactgcacccagtacatcagacacggcagcaagcccatgtacacccccgagcccgatatctgccacgagctgctgggacacgtgcccctgttcagcgacagaagcttcgcccagttcagccaggaaatcggcctggcctctctgggagcccccgacgagtatatcgagaagctggccaccatctactggttcaccgtggaattcggcctgtgcaagcagggcgacagcatcaaggcctacggcgctggcctgctgtccagctttggcgagctgcagtactgtctgagcgagaagcccaagctgctgcccctggaactggaaaagaccgccatccagaactacaccgtgaccgagttccagcccctgtactacgtggccgagagcttcaacgacgccaaagaaaaagtgcggaacttcgccgccaccatccccagaccattctccgtcagatacgacccctacacccagcggatcgaggtgctggacaacacacagcagctgaaaattctggccgactccatcaacagcgagatcggcatcctgtgcagcgccctgcagaagatcaagtgataagcatgcggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - DNA, 108 bp, Homo sapiens
  source 1..108: mol_type = other DNA
gaaacaagttacatcgaagacaactccaatcaaaatggtgctgtatctctgatattctcactcaaagaggaagttggtgccctggccaaggtcctgcgcttatttgag

SEQ ID NO: 27 - DNA, 49 bp, synthetic construct
  misc_feature 1..49: note = ENGINEERED SEQUENCE
  source 1..49: mol_type = other DNA
gaaacaagttacatcgaagacaactccaatcaaaacctgcttatttgag

SEQ ID NO: 28 - DNA, 109 bp, synthetic construct
  misc_feature 1..109: note = Engineered sequence
  source 1..109: mol_type = other DNA
gaaacaagttacatcgaagacaactccaatcaaaatggtgctgtatctctgatattctcactcaaagaggaagttggtgccctggtccaaggtcctgcgcttatttgag

SEQ ID NO: 29 - DNA, 109 bp, synthetic construct
  misc_feature 1..109: note = engineered sequence
  source 1..109: mol_type = other DNA
gaaacaagttacatcgaagacaactccaatcaaaaatggtgctgtatctctgatattctcactcaaagaggaagttggtgccctggccaaggtcctgcgcttatttgag

SEQ ID NO: 30 - DNA, 102 bp, synthetic construct
  misc_feature 1..102: note = engineered sequence
  source 1..102: mol_type = other DNA
gaaacaagttacatcgaagacaactccaatggtgctgtatctctgatattctcactcaaagaggaagttggtgccctggccaaggtcctgcgcttatttgag

SEQ ID NO: 31 - DNA, 108 bp, synthetic construct; identical to SEQ ID NO: 26
  misc_feature 1..108: note = Consensus sequence
  source 1..108: mol_type = other DNA